(19) United States
(12) Patent Application Publication
(10) Pub. No.: US 2004/0258652 A1
(43) Pub. Date: Dec. 23, 2004

(54) Hair treatment compositions and hair after-treatment compositions for protection against damage by chemical treatment and for the repair of already damaged hair comprising as active substances alkylguanidine compounds

(75) Inventors: Matthias Pascaly, Essen (DE); Nicole Flintrop, Duisburg (DE); Burghard Gruning, Essen (DE); Ursula MacZkiewitz, Essen (DE); Peter Lersch, Dinslaken (DE); Peter Muss, Essen (DE); Christian Weitemeyer, Essen (DE)

Correspondence Address: SCULLY SCOTT MURPHY & PRESSER, PC, 400 GARDEN CITY PLAZA, GARDEN CITY, NY, 11530

(73) Assignee: GOLDSCHMIDT AG, ESSEN (DE)

(21) Appl. No.: 10/858,945

(22) Filed: Jun. 2, 2004

(30) Foreign Application Priority Data
Jun. 18, 2003 (EP) 03013799.6

(51) Int. Cl. A61K 7/06; A61K 7/075; A61K 7/08

(52) U.S. Cl. 424/70.27

(57) ABSTRACT

Hair treatment compositions and hair after-treatment compositions are provided for the prevention of damage by chemical treatment compositions and for the repair of already damaged hair, wherein, as active substances, at least one of the compounds of the general formulae (I) and/or (II) are additionally used.

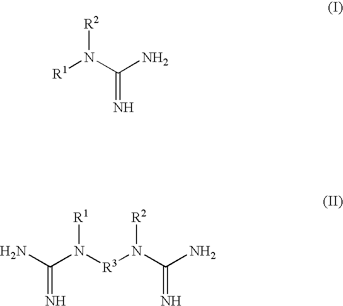

### FIELD OF THE INVENTION

The present invention relates to personal care products and more particularly to a hair treatment or after-treatment composition that is employed for the prevention of damage by chemical treatment compositions and for the repair of previously damaged hair. The hair treatment and hair after-treatment compositions of the present invention include at least an alkylguanidine compound and/or their salt conjugates.

### BACKGROUND OF THE INVENTION

Human hair is exposed daily to all sorts of influences. In addition to mechanical stresses due to brushing, combing, putting up or tying back, the hair is also attacked by environmental influences such as, for example, strong UV radiation, cold, wind and water. The physiological status (e.g., the age, and health) of the particular person also influences the damage to the keratinic fibers.

Treatment with chemical agents also changes the structure and surface properties of the hair. Methods such as, for example, permanent waving, bleaching, dyeing, tinting, smoothing, and etc., as well as frequent washing with aggressive surfactants contribute to more or less severe damage being caused to the hair structure. Thus, for example, during permanent waving both the cortex and the cuticle of the hair is attacked. The disulfide bridges of the cystine are broken open by the reduction step and partly oxidized to cysteic acid in the subsequent oxidation step.

With respect to bleaching, not only the melanine is destroyed, but also about 15 to 25% by weight of the disulfide bonds of the cystine are oxidized on mild bleaching. With excessive bleaching, up to 45% by weight of the disulfide bonds of cystine are oxidized (K. F. de Polo, A Short Textbook of Cosmetology, 2000, Verlag für chemische Industrie, H. Ziolkowsky GmbH).

Thus, disadvantageous mechanical properties for the hair result from chemical treatments, frequent washing or UV irradiation. It becomes brittle, dry, lusterless, porous and difficult to comb. Moreover, the hair loses moistness, elasticity and especially mechanical resistivity and tensile strength. This is seen in a significant decrease in the tensile powers of expansion and the breaking forces in the case of wet hair. Moreover, the hair is less resistant to further damage by chemicals, surfactants and environmental influences than healthy hair.

For the repair of hair damaged in this way, there are special preparations, such as, for example, hair rinses, hair tonics, shampoos, leave-in conditioners, and etc., which, can especially improve the combability, the handle and the luster of damaged hair. Commercial haircare compositions of this type mainly contain alkylammonium-based cationic surfactants, polymers, waxes or oils. The efficacy of these compounds can be attributed to an electrostatic interaction of the cationic quat groups or to a hydrophobization of the hair surface. A (bio)chemical repair of the hair is, however, not achieved by these compounds.

Great attention has been paid for a long time to the specific problem that in the case of hair the mechanical resistivity is greatly reduced by damage.

The use of creatine is known, for example, in this connection. In DE-A-101 14 561 and DE-A-101 196 08, the use of creatine compounds in compositions for hardening, strengthening, restructuring or increasing luster, volume or combability of keratinic fibers, in particular human hair, is described.

A disadvantage in the use of creatine is the fact that creatine can only be formulated via the aqueous phase and it is always in equilibrium with creatinine in aqueous solutions and is thus no longer available as an active substance for the hair.

Against this background, it is of great importance to identify further novel substances which have a structure similar to creatine and/or achieve a similar physiological action or alternatively assist and increase the action of creatine.

There is thus a need for active ingredients for hair treatment compositions and hair after-treatment compositions which can be employed in a versatile manner, which improve the mechanical resistivity of the hair, protect the hair against further damage to the hair structure and minimize the structural damage to the hair already caused, or produced by environmental influences and shape- and color-imparting treatments.

### SUMMARY OF THE INVENTION

It is an object of the present invention to make available such an active ingredient, which is able to improve both the mechanical resistivity of damaged hair, and to protect the hair from damage due to chemical treatment or exogenous factors such as smoking, smog, reactive oxygen species, free radicals and, in particular, light-related damage.

It has now surprisingly been found that simple alkylguanidine compounds and their acid conjugates in preparations for the treatment and after-treatment of the hair fulfill all these desired criteria.

The present invention thus provides the use of alkyl-guanidine compounds and/or their salt conjugates in hair treatment compositions and hair after-treatment compositions for the prevention of damage by chemical treatment compositions and for the repair of already damaged hair, wherein, as active substances, at least one of the compounds of the general formulae (I) and/or (II)

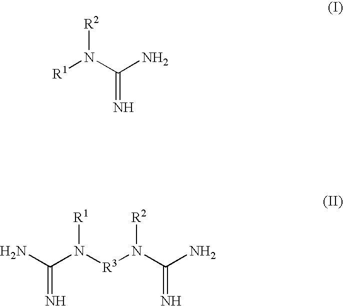

and/or their salts or hydrates, in which

R¹,R² a) independently of one another are H, an optionally branched hydrocarbon radical optionally containing double bonds, hydroxy-alkyl, alkyloxy, carboxyalkyl radicals, having 2 to 30 C atoms, preferably 4 to 22, in particular 8 to 12, C atoms, or

b) where the radicals can also have alicyclic or heterocyclic components, saturated, unsaturated or aromatic, having a ring size of 3 to 10 atoms, preferably from 4 to 6 atoms, which can carry further, saturated or unsaturated hydrocarbon substituents having 1 to 30 C atoms, preferably 4 to 22 C atoms, or

c) alkylamidoalkylene or alkyl ester alkylene radicals, which can further contain structural elements mentioned under a) and b) and the steal elements a), b), c) can be combined among one another and with one another,

R³ is alkylene, an optionally branched hydrocarbon radical having 1 to 30 C atoms, preferably 4 to 22 C atoms, optionally containing double bonds or alicyclic or heterocyclic components, which is saturated, unsaturated or aromatic, having a ring size of 3 to 10 atoms, preferably having a ring size of 4 to 6 atoms, or in which R¹ and R² in the element —N(R¹)—R³—(R²)N— can form a 5- to 8-membered ring, are present.

The present invention is also directed to alkylguanidines according to formula (I) and/or their salts, wherein at least one of the radicals R¹, R² is not hydrogen.

The present invention is further directed to alkyl-guanidines according to formula (II) and/or their salts, wherein R³ is hydrocarbon radicals, preferably alkylene radicals having 4 to 18, preferably 6 to 12, C atoms and R¹ and R² has the meaning indicated above and is preferably hydrogen.

### DETAILED DESCRIPTION OF THE INVENTION

The alkylguanidines used or additionally used according to the present invention, in particular straight-chain alkylguanidines, have both good stability and good formulability, produce a marked action even in low use concentrations, are not toxic, of natural origin or naturally identical, are very well tolerated by the hair and the scalp, have a high compatibility with other ingredients and can be incorporated into hair treatment compositions without problems. Additionally, the alkylguanidines employed in the present invention can also have a slight antimicrobial action.

The preparation of alkylguanidines is described in DE-C-506 282. In the process, alkylamines are guanidylated in an alcoholic solution using cyanamide in the presence of a protonic acid. The products are thus obtained as crystalline salts.

In DE-A-195 27 313, processes for the preparation of guanidine derivatives and their use as skin cosmetics are descried. This comprises (poly)alkylguanidines and alkoxy-guanidines, which on use produce a good skin sensation and have a moisture-donating and keratin-softening action.

In JP-A-2000247866, skin cosmetics are likewise described which have an excellent care effect and contain creatine and/or creatinine in combination with a further pharmaceutical active ingredient and/or a bioactive substance.

In JP-A-5-194150, the use of C₆₋₂₄-alkylguanidines in combination with fatty alcohols as hair conditioning agents is described.

The use of alkylguanidine derivatives is, according to known prior art, thus restricted to the use in, or as, conditioning agents for superficial treatments of the hair and in skin cosmetic formulations. Nothing is hitherto to be found about the surprising properties of this class of compound for the strengthening and protection of damaged hair and its repair.

Alkylguanidines of the general formula (I) and/or their salts and/or their hydrates have proven particularly suitable within the meaning of the present invention and are therefore preferred.

In principle, all cosmetically innocuous inorganic or organic mono- or polybasic acids are suitable for salt formulation, such as, for example, formic acid, acetic acid, propionic acid, heptanoic acid, caprylic acid, nonanoic acid, capric acid, undecanoic acid, lauric acid, myristic acid, palmitic acid, stearic acid, arachic acid, behenic acid, cyclopentanecarboxylic acid, cyclohexanecrboxylic acid, acrylic acid, methacrylic acid, vinylacetic acid, crotonic acid, 2-/3-/4-pentenoic acid, 2-/3-/4-/5-hexenoic acid, lauroleic acid, myristoleic acid, palmitoleic acid, oleic acid, gadoleic acid, sorbic acid, linoleic acid, linolenic acid, pivalic acid, ethoxyacetic acid, phenylacetic acid, lactic acid, 2-ethylhexanoic acid, oxalic acid, glycolic acid, malic acid, malonic acid, succinic acid, tartaric acid, glutaric acid, citric acid, adipic acid, pimelic acid, suberic acid, azelaic acid, sebacic acid, benzoic acid, o-/m-/p-toluic acid, salicylic acid, 3-/4-hydroxybenzoic acid, phthalic acids, or their completely or partly hydrogenated derivatives such as hexahydro- or tetrahydrophthalic acid, carbonic acid, phosphoric acid, hydrochloric acid, sulfuric acid and their mixtures, in particular lactic acid, tartaric acid, acetic acid and hydrochloric acid. Here, it is also possible within the meaning of the present invention to use both suitable guanidine derivatives in mixtures with one another and mixed salts.

Cosmetic preparations according to the present invention for the after-treatment, shaping and care of the hair are especially understood as meaning those hair treatment compositions which are used after a chemical treatment of the hair (hair after-treatment compositions) and chemical hair treatment compositions by means of which the hair structure is damaged and in which the damage can be minimized by the addition of alkylguanidine compounds (hair treatment compositions).

Alkylguanidines can generally be present here in a concentration of 0.01 to 10.0% by weight, preferably in a concentration of 0.1 to 5.0% by weight, in particular in a concentration of 0.1 to 2.0% by weight.

The hair after-treatment compositions are, for example, hair rinses, hair tonics, reviving compositions, leave-in conditioners, hair shampoos, two-in-one shampoos, setting formulations such as foam setting agents, hair sprays or hair dryer lotions, hair lotions, and hair tip fluids. They can be present as a gel, emulsion, solution, aerosol spray or foam, nonaerosol spray or foam.

The cosmetic preparations according to the present invention for the treatment of the hair after a chemical treatment have a pH of 3 to 7 and therefore preferably contain a water-soluble acid or a buffer mixture suitable, which stabilizes this pH.

The cosmetic preparations according to the present invention for the treatment of the hair after a chemical treatment can, in addition to alkylguanidine compounds, contain further components which are advantageous and/or customary for the particular application purpose. Thus, shampoos can contain, for example, 3 to 30% by weight of foaming anionic, zwitterionic, ampholytic and nonionic surfactants. Hair rinses and hair tonics can contain 0 to 10% by weight, preferably 0.5 to 5% by weight, of emulsifiers, 0 to 10% by weight, preferably 0.5 to 5% by weight, of consistency-imparting agents and 0 to 20% by weight of cosmetic oils of vegetable and synthetic origin, emollients, vitamin preparations and proteins. Shampoos, hair rinses, hair tonics and reviving agents preferably contain 0 to 8% by weight, preferably 0.1 to 5% by weight, of cationic surfactants and water-soluble polymers having quaternary ammonium groups for lowering the static chargeability and for improving combability, handle and luster.

The cationic surfactants are as a rule

quaternary ammonium compounds, such as, for example, alkyltrimethylammonium salts, dialkyldimethylammonium salts, trialkylmethylammonium salts and imidazolinium compounds. The long alkyl chains consist of a carbon chain having 10 to 22 C atoms, the counterions to the quaternary nitrogen are, for example, halides, sulfate, acetate, lactate, glycolate, nitrate or phosphate. Products are found on the market under the name Varisoft® 300, 432 CG, 442-100 P, BT 85 from Goldschmidt Rewo, Dehyquart® A from Henkel;

esterquats, such as are marketed under the name Dehyquart® F75 by Henkel or Armocare® VGH-70 by Akzo;

alkylamidoquats, such as are commercially available, for example, under the name Varisoft® PATC and RTM 50 from Goldschmidt Rewo.

The water-soluble polymers having quaternary ammonium groups are, for example,

cationic cellulose derivatives, such as are commercially obtainable under the name Celquat® H 100 and L 200 from National Starch or Polymer JR® from Amerchol,

polymeric dimethyldiallylammonium salts and their copolymers with esters and amides of acrylic acid and methacrylic acid. The products obtainable commercially under the name Merquat® 100 or Merquat® 550 from Calgon are examples of such cationic polymers,

copolymers of vinylpyrrolidone with quaternized derivatives of dialkyl aminoacrylate and methacrylate. Such compounds are commercially obtainable under the name Gafquat® 735 and Gafquat® 744 from ISP,

vinylpyrrolidone/vinylimidazolium methochloride copolymers, such as are supplied under the name Luviquat® FC 370, FC 550, FC 905 and HM-552 from BASF,

quaternized polyvinyl alcohol,

quaternized protein hydrolyzates of animal or vegetable origin based on keratin, collagen, elastin, wheat, rice, soybeans, milk, silk corn. Such products are marketed, for example, under the name Croquat® Wheat and Silk from Croda, Promois® W-32CAQ, Silk CAQ, WG CAQ from Seiwa Kasei or Quat-Coll® CDMA from Brooks,

guar hydroxypropyltrimethylammonium chloride,

aminofunctional polydimethylsiloxanes or hydroxylamino-modified silicones, such as the commercial products ABIL® Quat 3272 and ABIL® Quat 3474 from Goldschmidt, Dow Corning® 929 emulsion, Dow Corning® 939 from Dow Corning.

Setting agent formulations and other hair styling preparations customarily contain 0.1 to 5% by weight of film-forming polymers soluble in aqueous or aqueous-alcoholic media, optionally together with cationic surfactants or cationic polymers. Examples of film-forming agents are homo-polymers of vinylpyrrolidone, homopolymers of N-vinyl-formamide, copolymers of vinylpyrrolidone and vinyl acetate, terpolymers of vinylpyrrolidone, vinyl acetate and vinyl propionate, polyacrylamides, polyvinyl alcohols, high molecular weight polyethylene glycol or high molecular weight copolymers of ethylene glycol with propylene glycol, chitosan. These products are found on the market under the name Luviskol® K30, K60, K80, VA37E from BASF or PVP/VA E335 and PVP K30 from ISP.

Typical basic recipes for the respective applications are known prior art and are contained, for example, in the brochures of the manufacturers of the respective raw materials and active ingredients. These existing formulations can as a rule be adopted unchanged. If required for adaptation and optimization, the desired modifications, however, can be performed without complications by means of simple experiments.

A typical formulation for a hair rinse/hair tonic contains, for example:

a) 0.1 to 2% by weight of at least one of the compounds of the general formula (I) and/or (II),

b) 0.1 to 5% by weight of emulsifier,

c) 0.1 to 5% by weight of consistency-imparting agent,

d) 0.1 to 5% by weight of cationic surfactants and/or water-soluble polymers having quaternary ammonium groups,

e) 0 to 10% by weight of other cosmetic active ingredients, preservatives, and customary additives and excipients,

f) water to 100% by weight

A typical formulation for a hair shampoo contains, for example:

a) 0.1 to 2% by weight of at least one of the compounds of the general formula (I) and/or (II),

b) 3 to 30% by weight of foaming anionic, amphoteric, ampholytic or nonionic surfactants,

c) 0.1 to 5% by weight of cationic surfactants and/or water-soluble polymers having quaternary ammonium groups,

d) 0.1 to 6.0% by weight of thickener,

e) 0 to 10% by weight of other cosmetic active ingredients, opacifying agents, solvents and customary additives and excipients,

f) water to 100% by weight.

On account of their mild microbicidal action, the compounds of the general formula (I) and/or (II) can also be used or additionally used as active compounds in mild antidandruff formulations.

The preparations according to the present invention for chemical hair treatment are compositions for permanent shaping of the hair such as permanent wave and setting compositions or hair smoothing compositions, color-modifying agents such as bleaching agents, oxidation dyeing agents and tinting agents and shampoos based on direct-drawing dyes.

The preparations according to the present invention for the chemical treatment of the hair contain, in addition to alkylguanidine compounds and their derivatives, contain further components which are customarily employed for the respective application.

These are, in the case of a permanent wave solution, for example, 1 to 10% by weight of thioglycolic acid, thio-glycolic acid salts or esters. Permanent wave setting agents or bleaching agents preferably contain 2 to 10% by weight of oxidizing agent, such as, for example, potassium bromate, sodium bromate or hydrogen peroxide. Hair-smoothing agents are based on the use of strong bases or on reducing agents such as, for example, thioglycolic acid salts. Hair-coloring agents contain direct-drawing hair-coloring agents or oxidation dye precursors.

Finally, the preparations according to the present invention can contain cosmetic excipients and additives which are customary in such preparations. Such excipients are, for example, solubilizers such as ethanol, isopropanol, ethylene glycol, propylene glycol, glycerol and diethylene glycol, complexing agents such as EDTA, NTA, β-alaninediacetic acid and phosphonic acid, preservatives, antioxidants, fragrances, colorants for coloring the cosmetic preparation, opacifying agents such as latex, styrene/PVP and styrene-acrylamide copolymers, pearl luster agents such as ethylene glycol mono- and distearate and PEG-3 distearate, pigments, lightscreens, thickeners or propellants.

The alkylguanidine compounds can also be combined in the cosmetic preparations according to the present invention with other hair cosmetic active ingredients, such as, for example, ceramides, pseudoceramides, protein hydrolysates of vegetable or animal origin based on keratin, collagen, elastin, wheat, rice, soybeans, milk, silk, corn, antidandruff active ingredients such as piroctone olamine, zinc omadine and climbazole, sebostatics, vitamins, panthenol pyrrolidone-carboxylic acid, bisabolol, and plant extracts.

The hair treatment compositions according to the present invention are prepared in the customary manner, the alkylguanidine compounds being dissolved both in the aqueous and in the oil phase. The pH is preferably finally adjusted by addition of the acid and/or of the buffer mixture intended therefor.

Some preparation examples and recipes are given in the following text. These illustrate the subject of the present invention and do not restrict this.

### PREPARATION EXAMPLES

Preparation of octylguanidinium acetate and C₁₆₋₂₂-alkyl-guanidinium acetate:

30.7 g of octylamine or 70.8 g of C₁₆₋₂₂-alkylamine (0.2375 mol) was dissolved in 30 ml of n-butanol at elevated temperature with stirring. During the warning phase, 14.2 g of acetic acid (0.2365 mol) was added. After reaching the reaction temperature of 90° C., a solution of 10.0 g of cyanamide (0.2379 mol) in butanol (about 60 mol) was added dropwise over a period of 3 h and the batch was stir further at 90° C. for 3 h. After cooling, the solvent was stripped off under reduced pressure on a rotary evaporator. The product was suspended using acetone, filtered off and washed with diethyl ether. The final product was present as a crystalline colorless powder.

Octylguanidinium acetate: ¹³C-NMR, 100 MHz, CD₃OD, 25° C.: δ=181.0 (1C, COOH_{AcOH}), 159.3 (1C, C_{guanidinium gr.}),42.9 (1C, CH₂),33.4 (1C, CH₂),30.8 (1C, CH₂),30.4 (1C, CH₂),28.2 (1C, CH₂),24.8 (1C, CH₂),24.1 (1C, CH_{3,AcOH}), 14.9 (1C, CH₃); MALDI-TOF[m/z]: 172.2 (M+H⁺).

C₁₆₋₂₂-Alkylguanidinium acetate: ¹³C-NMR, 100 MHz, CD₃OD, 50 C: δ=178.5 (1C, COOH_{AcOH}), 157.1 (1C, C_{guanidine gr.}), 40.6 (1C, CH₂), 31.2 (1C, CH₂),28.9 (1C, CH₂),28.5 (1C, CH₂),28.5 (1C, CH₂),28.1 (1C, CH₂),25.9 (1C, CH₂),22.5 (1C, CH₂),21.8 (1C, CH_{3,AcOH}), 12.6 (1C, CH₃); MALDI-TOF[m/z]: 284.2 (C₁₆-guanidine+H⁺), 312.2 (C₁₈-guanidine+H⁺), 340.2 (C₂₀-guanidine+H⁺), 368.3 (C₂₂-guanidine+H⁺).

Preparation of octylguanidinium chloride:

15.4 g of octylamine (0.1194 mol) and 20 ml of n-butanol were initially introduced and warmed to 90° C. with stirring. During the warming phase, 11.0 g of hydrochloric acid (37%; 0.1137 mol) was added. After reaching the reaction temperature of 90° C., over a period of 3 h a solution of 5.0 g of cyanamide (0.1308 mol) in butanol (about 60 ml) was added dropwise and the batch was stirred further at 90° C. for 4 h. After cooling, the solvent was stripped off on a rotary evaporator under reduced pressure. The product was dissolved in H₂O and separated off using NaCl solution. Drying of the product was carried out by addition of ethanol and subsequent distillation. The final product was a highly viscous colorless liquid.

¹³C-NMR, 100 MHz, CD₃OD, 25° C.: δ=158.6 (1C, C_{guanidinium gr.}),42.9 (1C, CH₂),42.8 (1C, CH₂),33.0 (1C, CH₂),30.4 (1C, CH₂),29.9 (1C, CH₂),27.8 (1C, CH₂),23.8 (1C, CH₂),14.8 (1C, CH₃); MALDI-TOF[m/z]: 172.2 (M+H⁺), 192.9 (M+Na⁺).

Preparation of octylguanidinium lactate:

15.4 g of octylamine (0.1194 mol) and 20 ml of n-butanol were introduced into a multinecked round-bottomed a and warmed to 90° C. with stirring. During the warming phase, 10.2 g of lactic acid (0.1137 mol) was added. After reaching the reaction temperature of 90° C., over a period of 3 h a solution of 5.0 g of cyanamide (0.1194 mol) in butanol (about 60 ml) was added dropwise and the batch was stirred further at 90° C. for 3 h. After cooling, the solvent was stripped off on a rotary evaporator under reduced pressure. The product was suspended using acetone, filtered off and washed with diethyl ether. The final product was present as a crystalline colorless powder.

¹³C-NMR, 100 MHz, CD₃OD, 20° C.: δ=182.4 (1C, COOH_{lactic acid}),158.7 (1C, C_{guanidinium gr.}) 69.6 (1C, COH_{lactic acid}),42.5 (1C, CH₂),33.0 (1C, CH₂),30.34 (1C, CH₂),30.0 (1C, CH₂),27.8 (1C, CH₂),23.7 (1C, CH₂),21.7 (1C, CH_{3,lactic acid}),14.5 (CH₃), MALDI-TOF[m/z]: 172.1 (M+H⁺).

Preparation of hexane-1,6-diguanidinium acetate:

36.27 g (0.312 mol) of 1,6 diaminohexane, 37.49 g (0.624 mol) of acetic acid and 180 ml of n-butanol were introduced and warmed to 90° C. The cyanamide was dissolved in 80 ml of 1-butanol and continuously added dropwise at 90° C. over the course of 3 hours. After completion of the addition, the mixture was allowed to subsequently react at 90° C. for 4 hours. The solvent was stripped off on the rotary evaporator. The residue was then recrystallized from water, washed twice with 50 ml of acetone each time and the product was dried at 60° C. in vacuo. The final product was obtained as a crystalline pale beige powder.

¹³C-NMR, 100 MHz, D₂O, 20° C.: δ=181.1 (2C, COOH_{AcOH}), 156.6 (2C, C_{guanidinium gr.}),40.9 (2C, CH₂),27.6 (2C, CH₂),25.3 (2C, CH₂),23.2 (2C, CH_{3,AcOH}); MALDI-TOF[m/z]: 201.2 (M+H⁺).

Preparation of N,N-dibutylguanidinium acetate:

55.86 g (0.432 mol) of dibutylamine, 25.96 g (0.432 mol) of acetic acid and 100 ml of 1-butanol were introduced and warmed to 90° C. The cyanamide was dissolved in 60 ml of n-butanol and continuously added dropwise at 90° C. over the course of 3 hours. After completion of the addition, the mixture was allowed to subsequently react at 90° C. for 4 hours. At 76° C., the precipitated salt was filtered off and washed twice with 50 ml of acetone each time. The product was dried at 60° C. in vacuo on the rotary evaporator, and the final product was obtained as a colorless crystalline substance.

¹³C-NMR, 100 MHz, ethanol-d₆, 20° C.: δ=178.8 (1C, COOH_{AcOH}), 155.9 (1C, C_{guanidinium gr.}) 48.1 (2C, CH₂), 29.0 (2C, CH₂), 23.3 (1C, CH_{3,AcOH}), 19.2 (2C, CH₂), 12.9 (2C, CH₂); MALDI-TOF[m/z]: 172.2 (M+H⁺).

Recipes:

Application Testing:

Hair used:

Euro hair (hair from Europeans), left natural.

Preliminary Damage to the Hair

1×each permanent waving and bleaching using commercially available products.

Treatment of the damaged hair:

The damaged hair was treated with the exemplary formulations.

TABLE 1

| | | Hair rinse B |
|---|---|---|
| | | (not according to the |
| | Hair rinse A | present invention) |
| Alkylguanidinium compound [% by | 2.0 | — |
| weight] | | |
| Ceteareth-25 [% by weight] | 0.5 | 0.5 |
| Cetearyl alcohol | 2.0 | 2.0 |
| [% by weight] | | |
| HCl | to pH = 5 | to pH = 5 |
| Water [% by weight] | to 100 | to 100 |

The improvement in the mechanical resistivity of damaged hair due to alkylguanidinium compounds was determined by pairwise comparison of individual hairs before and after treatment with the test formulation.

For each test formulation, 30 damp individual hairs were measured. The experimental procedure was as follows:

1. The predamaged hairs were wetted with water and using the fully automatic device Mtt670 from DiaStron the force was measured which was necessary for 15% extension.

2. For recovery, the hairs were immersed for at least 2 h in a water bath.

3. The hairs were then immersed for 30 min in the test formulation and then each hair was rinsed for about 7 s under running water.

4. The hairs were left overnight (=12 h) to dry in air.

5. The force which was necessary in order to extend the treated hair by 15% was again measured as described above, after wetting with water.

6. The difference between the 15% extension forces before and after treatment with the test formulation was calculated and used as a measure of the improvement in the mechanical resistivity of the damaged hairs by the alkylguanidine compounds.

For statistical assessment of the measurements, the t-test was used for pairwise comparison both of the measurements before and after treatment and the measurements of the treated hair were compared with the result of the placebo treatment. A statement can thus be made about the statistical certainty of the measurements (for this see also: R. E. Kaiser, J. A. Mühlbauer, “Elementare Tests zur Beurteilung von Meβdaten” [Elementary Tests for the Assessment of Measurements], B. I. Wissenschaftsverlag, Mannheim 1983). It was assumed with a statistical certainty of >95% that a significant difference exists, >99% means that the measurements differ highly significantly, >99.9 that they differ very highly significantly.

TABLE 2A
Determination of the force for the extension of individual hairs
by 15% before and after their treatment (at least 30
independent measurements):

| Test formulations | | | | |
|---|---|---|---|---|
| according to | | | | t-test: |
| Tab. 1 with novel | Force | | | comparison |
| compound of formula (I) | 15%_{after} | | | with mean |
| where R¹ = H | force | | | value of |
| present as the | 15%_{before} | | Pairwise | placebo |
| acetic acid salt | [mN] | σ | t-test [%] | [%] |
| HSP B | 0.49 | 2.41 | 72.9 | |
| HSP A where R² = C₄ | 1.36 | 1.62 | 100.0 | 89.0 |
| HSP A where R² = C₆ | 1.72 | 1.61 | 100.0 | 97.8 |
| HSP A where R² = C₈ | 3.51 | 1.64 | 100.0 | 100.0 |
| HSP A where R² = C₁₀ | 3.00 | 1.83 | 100.0 | 100.0 |
| HSP A where R² = C₁₂ | 3.30 | 1.64 | 100.0 | 100.0 |
| HSP A where R² = C₁₆ | 1.89 | 1.33 | 100.0 | 99.3 |
| HSP A where R² = C₁₈ | 1.80 | 1.45 | 100.0 | 98.7 |
| HSP A where R² = C₂₂ | 0.94 | 2.21 | 97.3 | 54.0 |

HSP: Hair rinse
Force 15%_{after:} 15% extension force after treatment with the test formulation.
Force 15%_{before:} 15% extension force before treatment with the test formulation.
σ: Standard deviation

The alkylguanidine compounds were thus able to increase the 15% extension forces of damaged hair highly significantly, i.e., they surprisingly and markedly improved the mechanical resistivity of damaged hair.

TABLE 2B
Determination of the force for the extension of individual hairs by 15%
before and after their treatment (at least 30 independent
measurements) by novel compounds of the formulae (I) and
(II) having different radicals R¹, R² and R³

| | Force | | | t-test: |
|---|---|---|---|---|
| | 15%_{after} | | | comparison with |
| | force | | | mean value of |
| Test formulations according to Tab. 1 | 15%_{before} | | Pair-wise | placebo |
| containing novel compound | [mN] | σ | t-test [%] | [%] |
| HSP B | 0.49 | 2.41 | 72.9 | |
| HSP with compounds according to | | | | |
| formula (I): | | | | |
| HSP A where R¹ = H; R² = C₆; lactate | 1.36 | 1.96 | 99.9 | 97.8 |
| HSP A where R¹ = H; R² = C₈; lactate | 3.65 | 2.27 | 100 | 95.1 |
| HSP A where R¹ = H; R² = C₁₀; | 3.44 | 2.07 | 100 | 90.8 |
| lactate | | | | |
| HSP A where R¹ = H; R² = C₁₂; | 4.38 | 2.26 | 100 | 99.9 |
| lactate | | | | |
| HSP A where R¹ = H; R² = oleyl; | 6.33 | 3.63 | 100 | 100 |
| acetate | | | | |
| HSP A where R¹ = H; R² = coco; | 4.79 | 2.78 | 100 | 99.9 |
| acetate | | | | |
| HSP A where R¹ = H; R² = phenyl; | 0.66 | 2.94 | 77.2 | 99.8 |
| carbonate | | | | |
| HSP A where R¹ = H; R² = benzyl; | 1.58 | 2.29 | 99.9 | 15 |
| acetate | | | | |
| HSP A where R¹ = C₄; R² = C₄; | 2.16 | 3.12 | 99.9 | 50 |
| acetate | | | | |
| HSP A where R¹ = C₈; R² = C₈; | 4.54 | 2.58 | 100 | 100 |
| acetate | | | | |
| HSP A where R¹ = H; | 3.59 | 3.06 | 100 | 62 |
| R² = H(OCH₂CH₂)₁₄; acetate | | | | |
| HSP A where R¹ = CH₂CH₂OH; | 3.64 | 2.69 | 100 | 71 |
| R² = palmoylethylamide; acetate | | | | |
| HSP A where R¹ = H; R² = 6-caproic | 2.06 | 1.69 | 100 | 83 |
| acid butylamide; phosphate | | | | |
| HSP A where R¹ = H; R² = alkyl- | 2.85 | 1.85 | 100 | 47 |
| dimethicone; acetate | | | | |
| HSP A where | 0.56 | 2.57 | 75 | 99.95 |
| R¹ + R² + N = | | | | |
| (—(CH₂)₂—O—(CH₂)₂—); | | | | |
| acetate | | | | |
| HSP A containing arginine | 0.358 | 2.65 | 49.3 | 99.6 |
| HSP containing compounds | | | | |
| according to formula (II): | | | | |
| HSP A where R¹, R² = H; R³ = C₆; | 3.45 | 2.4 | 100 | 99.5 |
| acetate | | | | |
| HSP A where R¹, R² = H; R³ = C₁₂; | 3.6 | 2.84 | 100 | 90 |
| acetate | | | | |

HSP: Hair rinse
Force 15%_{after:} 15% extension force afer treatment with the test formulation.
Force 15%_{before:} 15% extension force before treatment with the test formulation.
σ: Standard deviation

As listed in Table 2b, the very different types of guanidine compounds were able to increase the 15% extension forces of damaged hair highly significantly, i.e., they surprisingly and markedly improved the mechanical resistivity of damaged hair. In contrast to the novel compounds, arginine improved the extension forces of damaged hair only insignificantly.

Protection of the Hair from Damage by Chemical Agents:

The more severely damaged the hair, e.g., by permanent waving or bleaching, the higher the concentration of water-soluble protein fragments/peptides in the hair which can be extracted with water or surfactants. Extraction with surfactants simulates the washing of the hair by means of a shampoo. Also during hair washing, damaged hair loses more peptides than healthy hair.

For illustration, the hair was predamaged. For this, the hair was permanently waved and bleached 1×each

TABLE 3
Test formulation of a hair rinse containing 2% of active ingredient:

| | |
|---|---|
| TEGINACID ® C (emulsifier) | 0.5% |
| TEGO ® Alkanol 1618 (consistency- | 2.0% |
| imparting agent) | |
| Alkylguanidine compound | 2.0% AS |
| Preservative (CA24) | q.s. |
| HCl | pH = 5.0 |
| Water | to 100.0% |

The predamaged strands of hair (weight: about 4 g) were immersed in the test formulation (time of action: 30 min) and then rinsed off for 1 min under running tap water (T=35° C.).

They were then left to dry in the air for 12 hours.

Determination of the Protein Loss:

About 0.5 g of hair+about 15 ml of Na lauryl ether sulfate (8%) were shaken at room temperature for 3 h. The solution was subsequently decanted and centrifuged. The protein concentration of the solution was then determined photometrically by measurement of the UV absorption at 295 nm. A keratin hydrolysate was used as a standard (M=1000, Promois WK-H, Seiwa Kasei).

TABLE 4
Determination of the protein loss based on 1 g of hair
(at least three independent measurements):

| Formulation according to Tab. 3 | | |
|---|---|---|
| containing novel compound of | | |
| formula (I) where R¹ = H | Protein loss | |
| present as the acetic acid salt | [mg/g of hair] | σ |
| Virgin | 13.7 | 0.39 |
| damaged | 35.0 | 0.87 |
| Placebo | 28.8 | 0.62 |
| R² = C₄ | 20.3 | 0.12 |
| R² = C₆ | 26.0 | 1.25 |
| R² = C₈ | 26.8 | 0.43 |
| R² = C₁₀ | 26.4 | 0.79 |
| R² = C₁₂ | 23.5 | 0.67 |
| R² = C₁₆ | 25.9 | 0.79 |
| R² = C₁₈ | 29.8 | 0.54 |
| R² = C₂₂ | 28.9 | 0.84 |

σ = Standard deviation

It is recognized that the alkylguanidinium compounds reduced the protein loss of the damaged hair, the short-chain compounds in particular showed a very good protective action here.

While the present invention has been particularly shown and described with respect to preferred embodiments thereof, it will be understood by those skilled in the at that the foregoing and other changes in forms and details may be made without departing from the scope and spirit of the present invention. It is therefore intended that the present invention not be limited to the exact forms and details described and illustrated, but fall within the scope of the appended claims.

What we claim is:

1. A hair treatment composition or hair after-treatment composition for the prevention of damage by chemical treatment compositions or exogenous factors, for the repair of already damaged hair and for strengthening the hair, comprising, as active substances, at least one compound of general formulae (I), (II), or a mixture of (I) and (II) 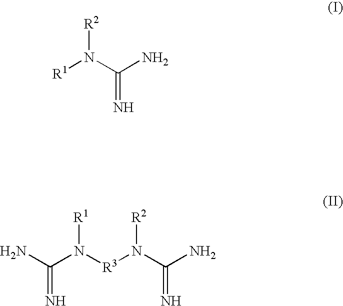
and/or their salts or hydrates, in which
R¹, R² a) independently of one another are H, a hydrocarbon radical, hydroxy-alkyl, alkyloxy, carboxyalkyl radicals, having 2 to 30 C atoms, or
b) where the radicals can also have alicyclic or heterocyclic components, saturated, unsaturated or aromatic, having a ring size of 3 to 10 atoms, which can carry further, saturated or unsaturated hydrocarbon substituents having 1 to 30 C atoms, or
c) alkylamidoalkylene or alkyl ester alkylene radicals, which can further contain structural elements mentioned under a) and b), and the structural elements a), b), c) can be combined among one another and with one another,
R³ is alkylene, an hydrocarbon radical having 1 to 30 C atoms, or alicyclic or heterocyclic components, which is saturated, unsaturated or aromatic, having a ring size of 3 to 10 atoms, or in which R¹ and R² in the element —N(R¹)—R³—(R²)N— form a 5- to 8-membered ring.

2. The hair treatment composition or hair after-treatment composition as claimed in claim 1, where the compound is a compound of formula (I) and/or its salts, wherein at least one of the radicals R¹, R² is not hydrogen.

3. The hair treatment composition or hair after-treatment composition as claimed in claim 1, wherein the compound is a compound of formula (II) and/or its salts, wherein R³ is a hydrocarbon radical and R¹ and R² have the meaning indicated above.

4. The hair treatment composition or hair after-treatment composition as claimed in claim 3, wherein R³ is an alkylene radical containing 4 to 18 C atoms.

5. The hair treatment composition or hair after-treatment composition as claimed in claim 1, wherein, as salts, at least one of the acids selected from the group consisting of formic acid, acetic acid, propionic acid, heptanoic acid, caprylic acid, nonanoic acid, capric acid, undecanoic acid, lauric acid, myristic acid, palmitic acid, stearic acid, arachic acid, behenic acid, cyclopentanecarboxylic acid, cyclohexanecarboxylic acid, acrylic acid, methacrylic acid, vinylacetic acid, crotonic acid, 2-/3-/4-pentenoic acid, 2-/3-/4-/5-hexenoic acid, lauroleic acid, myristoleic acid, palmitoleic acid, oleic acid, gadoleic acid, sorbic acid, linoleic acid, linolenic acid, pivalic acid, ethoxyacetic acid, phenylacetic acid, lactic acid, 2-ethylhexanoic acid, oxalic acid, glycolic acid, malic acid, malonic acid, succinic acid, tartaric acid, glutaric acid, citric acid, adipic acid, pimelic acid, suberic acid, azelaic acid, sebacic acid, benzoic acid, o-/m-/p-toluic acid, salicylic acid, 3-/4-hydroxybenzoic acid, phthalic acids, or their completely or partly hydrogenated derivatives such as hexahydro or tetrahydrophthalic acid, carbonic acid, phosphoric acid, hydrochloric acid, sulfuric acid and their mixtures, in particular lactic acid, tartaric acid, acetic acid and hydrochloric acid is additionally used.

6. The hair treatment composition or hair after-treatment composition as claimed in claim 1, which contains 0.05 to 10.0% by weight of the compounds of the general formulae (I) and/or (II) and/or their salts and/or their hydrates.

7. The hair treatment composition or hair after-treatment composition as claimed in claim 1, further comprising 0 to 10% by weight of one or more emulsifiers, 0 to 10% by weight of one or more consistency-imparting agents, 0 to 10% by weight of one or more, preferably cationic, surfactants 0 to 20% by weight of one or more cosmetic oils or emollients, and customary excipients and additives in customary concentrations, which contains 0.05 to 10.00% by weight of the compounds of the general formulae (I) and/or (II) and/or their salts and/or their hydrates.

8. The hair treatment composition or hair after-treatment composition as claimed in claim 7, further comprising one or more hair cosmetic active ingredients selected from the group consisting of the protein hydrolysates of vegetable or animal origin based on keratin, collagen, elastin, wheat, rice, soybeans, milk, silk, corn; antidandruff active ingredients such as piroctone olamine, zinc omadine and climbazole, sebo-statics; vitamins, panthenol, pyrrolidonecarboxylic acid, bisabolol, plant extracts, creatine, and ceramides.

9. A hair shampoo, leave-in formulation comprising at least the hair treatment or hair after-treatment composition of claim 1.

10. A cosmetic formulation having antidandruff action comprising, as an active component the hair treatment or hair after-treatment composition of claim 1.
